(19) United States
(12) Patent Application Publication
(10) Pub. No.: US 2024/0170118 A1
(43) Pub. Date: May 23, 2024

(54) HOLISTIC PERSONALITY ASSESSMENT

(71) Applicant: Veritas Index Institute, Wilmington, DE (US)

(72) Inventor: Steven Lam, Lexington, MA (US)

(21) Appl. No.: 18/512,893

(22) Filed: Nov. 17, 2023

Related U.S. Application Data
(60) Provisional application No. 63/426,503, filed on Nov. 18, 2022.

(51) Int. Cl. G16H 20/00; G16H 40/67

(52) U.S. Cl. CPC G16H 20/00; G16H 40/67

(57) ABSTRACT

The present disclosure is for a system and a method for evaluating cognitive performance capabilities as a function of emotional intelligence. The present invention provides a way to more accurately and objectively assess an individual's true emotional intelligence by adjusting an individual's self-reported and often biased personality assessment by accounting for lifestyle factors and cognitive task performance factors indicative of an individual's true emotional intelligence. The adjusted emotional intelligence aids in better evaluating an individual's or team's productivity capacity allowing for better planning and allocation of tasks to improve overall productivity and may serve to reduce burnout.

### CROSS-REFERENCE TO RELATED APPLICATIONS

This application claims the benefit of U.S. Provisional Application 63/426,503, filed Nov. 18, 2022, titled “HOLISTIC PERSONALITY ASSESSMENT,” which is herein incorporated by reference in its entirety.

### BACKGROUND

Emotional intelligence may provide an important indicator of both individual and team productivity as cognitive abilities (and thus task performance) change as a result of true emotional intelligence and lifestyle factors. However, assessing an individual's emotional intelligence is a difficult task. A common approach involves questionnaires, however these can be biased by the way a person views themself which may not be an accurate indicator of true emotional intelligence. Moreover, changes in cognitive status as it pertains to emotional intelligence and day to day lifestyle is not readily apparent on a day to day or week to week basis. This poses problems for organizations, teams, etc. trying to optimize performance and best assign roles and responsibilities to the individual's best suited for such. Even if someone recognizes changes in their or a colleague's cognitive capacity, it is not readily apparent how these affect performance and to what extent as each individual may have different performance outcomes based on their differing emotional intelligence characteristics. While current questionnaire based approaches to determining emotional intelligence may provide some degree of personality assessment, an individual's true emotional intelligence is often reflected in cognitive task performance and lifestyle factors. Thus, reliance on self-reported EI is not sufficiently reliable to evaluate cognitive capabilities for given task categories or forecast where performance may be headed. Moreover, there is currently no well-established means for determining emotional intelligence from cognitive task performance and lifestyle factors. Furthermore, there is currently no well established relationship between cognitive function capabilities and an objective emotional intelligence assessment which is more indicative of true emotional intelligence.

In addition, the difference between an individual or team's cognitive ability and emotional intelligence may provide valuable insight into burnout and productivity metrics. There is a need for a comprehensive and ongoing analysis of an individual's cognitive state with respect to their emotional intelligence. Furthermore, there is a need for a team based comprehensive and ongoing analysis of cognitive state relative to emotional intelligence for identifying productivity measures.

Different cognitive task performance may be significantly affected by personality characteristics. Emotional intelligence may impact individual performance under various circumstantial stressors. Collectively a team or group performance may be affected not only by each individual's performance but also the position of each individual, the associated cognitive tasks they face and how their emotional intelligence affects their ability to perform those cognitive tasks. Thus team performance may be due, at least in part, to a mismatch between the emotional intelligence of an individual and their corresponding responsibilities to the team due to their role. There is a need for a team based analysis of cognitive tasks and cognitive task importance in association with emotional intelligence characteristics of the critical cognitive task performers.

Often, productivity analysis may fail to consider an individual's personality or mental capacity for the cognitive tasks/responsibilities associated with their role or how those cognitive tasks will affect the individual over time. Forecasting productivity based on personality assessment may provide valuable insight into projected productivity as well as tracking productivity over time and finding ways to optimize team performance based on emotional intelligence characteristics of team members. Furthermore, productivity analysis may fail to consider both individual and team burnout which can impact an individual's ability to handle different scenarios. Monitoring over time may allow for adaptations in assignment of team responsibilities to account for burnout and thus lead to better overall team performance.

### SUMMARY

The present invention addresses the above issues by providing systems and methods for assessing emotional intelligence and subtle changes in emotional intelligence over time. By tracking lifestyle characteristics such as daily behaviors associated with diet, sleep, exercise, staying on schedule, etc. in addition to assessing cognitive task performance, an individual's true emotional intelligence characteristics are revealed. While questionnaires may serve as a good starting point for measuring emotional intelligence, the present invention overcomes any inherent bias present in questionnaire assessments by adjusting emotional intelligence ratings over time based on lifestyle factors which are a more accurate indicator of a person's true emotional intelligence characteristics. For example, a person may view themselves as a motivated, punctual individual and report such on a questionnaire, however monitoring their behavior and lifestyle factors over time can serve to confirm this or show that they tend to lack motivation and are frequently late or behind schedule. Furthermore, as emotional intelligence tends to change subtly over time, monitoring lifestyle factors can serve to provide an indication of the long term trends and changes in an individual's emotional intelligence which may not be captured by questionnaire data alone.

The present invention derives at least one lifestyle metric from one or more of physiology data associated with lifestyle activities obtained from a wearable sensor and user reported lifestyle data reported by a user over a period of time (e.g. days, weeks, etc.). Cognitive performance metric(s) are derived from additional physiology data obtained from the wearable sensor as an individual is performing specified task performance activities. Emotional intelligence metric(s) are derived from user responses to a plurality of questions. The lifestyle metric(s), cognitive performance metric(s), and emotional intelligence metric(s) are combined to determine a cognitive function metric(s) indicative of an individual's cognitive capacity, a metric which cannot be determined via conventional approaches. Changes in cognitive function capacity over time can be detected as a result of lifestyle factors which can be weighted differently depending on at least one of emotional intelligence and cognitive task performance metrics again providing a metric which conventional technologies are unable to provide.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a system for holistic personality assessment in accordance with an exemplary embodiment of the invention.

FIG. 2 illustrates a system for rating performance in accordance with an exemplary embodiment of the present invention.

FIG. 3 illustrates an exemplary process for rating performance according to one embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

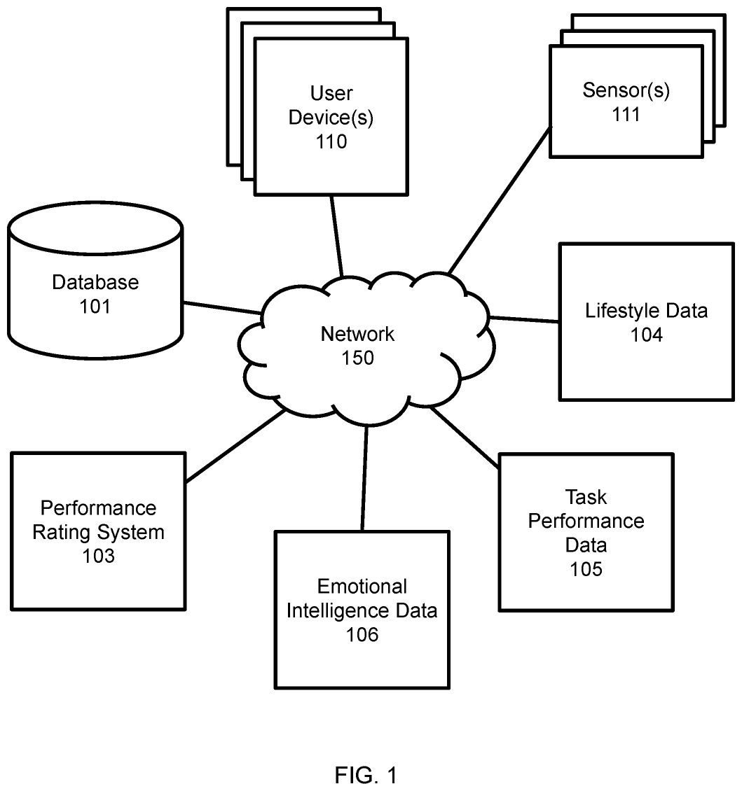

FIG. 1 illustrates an exemplary embodiment of a holistic personality assessment system according to one embodiment. The system includes user device(s) 110, sensor(s) 111, lifestyle data 104, cognitive task performance data 105, emotional intelligence data 106, database 101, and a network 150 over which the various systems communicate and interact. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same cognitive tasks on one or more other servers or computing devices without departing from the scope of the invention.

Performance rating system 103 is operable to obtain at least one of lifestyle data 104, cognitive task performance data 105, and emotional intelligence data 106 and analyze the obtained data to determine at least one metric associated with cognitive and/or emotional intelligence characteristics for each individual associated with the obtained data. Performance rating system 103 is operable to analyze the obtained data for a plurality of individuals, such as a group of individuals who are part of a team, work group, etc., and compute at least one metric associated with cognitive and/or emotional intelligence characteristics for the group. Performance rating system 103 is operable to compute at least one metric associated with at least one of a static performance metric, dynamic performance metric, and a forecasted or predicted performance metric. Performance rating system 103 is operable to determine at least one metric based on a difference between the cognitive characteristics and emotional intelligence characteristics of individuals and/or groups. In one aspect, this difference metric may provide an indication of burnout of an individual(s) and/or team or group of individuals. Performance rating system 103 may determine at least one of an individual performance capacity, a group performance capacity, an individual burnout metric, and a team burnout metric, any of the preceding determined as at least one of a static metric (e.g. for a given time period, such as a day, weck, etc.), a dynamic metric (e.g. as changes in the metrics occur over time), and a forecasted metric (e.g. predicting the trends associated with an individual or team, an endpoint of interest where the individual or team will end up if the trend continues, and a time frame until the endpoint of interest is achieved).

Lifestyle data 104 comprises at least one of active lifestyle data, passive lifestyle data, and biofeedback data. Active lifestyle data may comprise consciously made lifestyle decisions. Active lifestyle data may comprise at least one of user bedtime, diet quality, cardiovascular activity metrics, and emotional intelligence metrics. Passive lifestyle data may comprise subconscious lifestyle decisions, and may be at least partially affected by active lifestyle factors. Passive lifestyle data may comprise at least one of sleep duration, wakeup time, punctuality, time between dinner and sunset, time between bedtime and sunset, time between wakeup and sunset, and diet factors in association with bowel movement characteristics. Biofeedback data may comprise physiology characteristics and may be at least partially affected by active and/or passive lifestyle factors. Biofeedback data may comprise at least one of resting heart rate, REM (rapid eye movement) or deep sleep duration, urine color, and bowel movement characteristics.

cognitive task performance data 105 comprises biofeedback data associated with at least one cognitive task performed under at least one of a normal or baseline condition and a stimuli based condition (e.g. mental stimuli, emotional stimuli, physical stimuli). cognitive task performance data may comprise a change metric indicating the change in at least one metric associated with the biofeedback data between the baseline and stimuli conditions. For example, heart rate may be measured while a user is performing a cognitive task under a baseline condition and then while the user is performing the same cognitive task under a stimuli condition. A change in heart rate between the two conditions may be computed and used as a metric making up a portion of the cognitive task performance data. Cognitive task performance data may comprise cognitive task performance data associated with at least one of a plurality of cognitive tasks cach designed to assess a different emotional intelligence element. For example, cognitive tasks may comprise at least one of planning cognitive tasks, anticipation cognitive tasks, risk management cognitive tasks, focus cognitive tasks, memory cognitive tasks, and correlation cognitive tasks. The cognitive tasks may be designed to assess (either alone or based on a combination of cognitive task performances) at least one emotional intelligence category, including, but not limited to, at least one of motivation, self awareness, interpersonal skills, self regulation, adaptability, and facial recognition.

Emotional intelligence (EI) data 106 may comprise questionnaire data associated with a plurality of questions and associated user responses designed to assess various emotional intelligence characteristics. EI data may comprise two parts, including at least a first part and second part. The first part may comprise a plurality of questions (e.g. 200) used to establish an individual's baseline or standard emotional intelligence characteristics. The second part may comprise subsets of questions asked periodically over time in order to identify changes in an individual's mental status as it relates to changes in cognitive status and/or emotional intelligence changes over time. In one aspect, the emotional intelligence characteristics comprise a plurality of EI categories, including, but not limited to, at least one of motivation, self awareness, interpersonal skills, self regulation, adaptability, and facial recognition. The EI data 106 may comprise user responses obtained at a plurality of time intervals including, but not limited to, at least one of daily responses, weekly responses, monthly responses, responses obtained at multiple time periods throughout a day, responses obtained at multiple time periods across a plurality of days, responses obtained at multiple time periods across a week or plurality of weeks, responses obtained at multiple time periods across a month or plurality of months, etc. EI data may comprise responses to a subset of EI questions selected from a larger plurality of EI questions which are selectively presented to a user at planned (optionally recurring) time points. For example, a first subset of EI questions may be presented to a user on a first day, a second subset of EI questions presented on a second day, a third subset of EI questions presented on a third day, and so on. In one aspect, after a desired number of subsets of EI questions, the process may repeat beginning again with the first subset. Alternatively, each subset of questions may continually change over time. In one aspect, the EI questions are selected and/or presented in a randomized fashion without a particular designed sequence, planning or repetition.

Database 101 is operable to obtain at least one of the lifestyle data 104, cognitive task performance data 105, emotional intelligence data 106, data from user device(s) 110, data from sensor(s) 111, and data from performance rating system 103 and store the data for later access and processing. In one aspect, the database 101 stores the data in a standardized format to account for the fact that each of the different data sources may result in or provide data in different or non-standard formats. In one aspect, database 101 stores the data in such a way as to associate each data set with at least one of a corresponding user and a corresponding team or group the user is part of or otherwise associated.

Sensor(s) 111 are operable to provide at least one of lifestyle data 104 and cognitive task performance data 105. In one aspect, sensor(s) 111 comprise at least one wearable device, including, but not limited to, smart watches and other sensors capable of sensing at least one of physiology information and activity information associated with a user/wearer as would be apparent to one of ordinary skill in the art. Sensor(s) 111 may be operable to provide at least one of heart rate, blood pressure, body temperature, sleep information (e.g. sleep/wake times, sleep duration), and the like.

User device(s) 110 are operable to provide at least one of lifestyle factor data, cognitive task performance data, and emotional intelligence data for use by performance rating system 103 in the analysis described above. For example, user device(s) 110 are operable to provide response data related to at least one of emotional intelligence questions, diet, exercise, urine characteristics, bowel movement characteristics, etc.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links cach includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

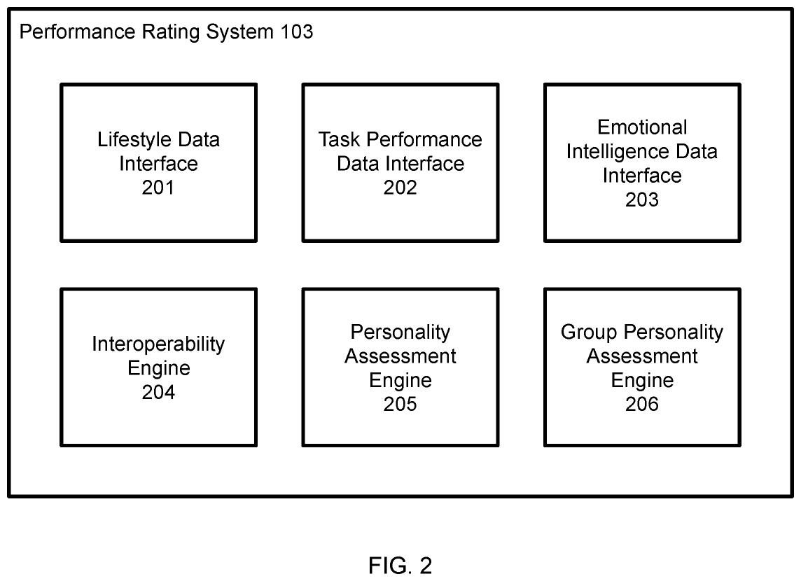

FIG. 2 illustrates an exemplary embodiment of the performance rating system 103. The performance rating system 103 comprises lifestyle data interface 201, cognitive task performance data interface 202, emotional intelligence data interface 203, interoperability engine 204, personality assessment engine 205, and group personality assessment engine 206. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

Lifestyle data interface 201, cognitive task performance data interface 202, and emotional intelligence data interface 203 are operable to obtain their respective data sets (as described in FIG. 1 above) via communication with at least one of user device(s) (e.g. user devices 110), sensor(s) (e.g. sensors 111), and database(s) (e.g. databases 101). Interfaces 201, 202, and 203 are operable to obtain data via a data push from other components or devices and/or via a data pull or request for data from other components or devices. In one aspect, the data interfaces obtain data in real-time as the data becomes available (e.g. as a user enters data via a user device).

Interoperability engine 204 is operable to convert obtained data from a non-standard format to a standardized format. As each data set may come from a different data source (e.g. different user devices, different sensors, etc.) and each source may provide the data in a different format, interoperability engine 204 converts data from different sources to a standardized format, as necessary, for further processing and analysis.

Personality assessment engine 205 is operable to compute at least one metric associated with an individual's cognitive, emotional intelligence and/or lifestyle characteristics. In one aspect. EI rating engine 205 uses a large dataset (e.g. 200 questions) to establish initial EI characteristics for an individual. In general an individual's EI characteristics do not change substantially over a short term basis (e.g. day to day) and thus provides a point of comparison against which cognitive and lifestyle status (which does tend to change on a day to day basis) can be compared. EI rating engine 205 is operable to compute a metric for each of a plurality of EI characteristics. Exemplary EI characteristics include, but are not limited to, motivation, self awareness, interpersonal skills, self regulation, adaptability, and facial recognition. In one aspect, EI rating engine 205 computes a plurality of cognitive characteristics for each individual. Exemplary cognitive characteristics comprise at least one of planning, anticipation, risk management, focus, memory, and connectivity. In one aspect, cognitive characteristics are determined based on a combination of the lifestyle data, cognitive task performance data, and emotional intelligence data. Since cognitive characteristics are based on lifestyle data, they tend to change over time. EI rating engine 205 is operable to obtain and analyze at least one of the lifestyle data, cognitive task performance data, and emotional intelligence data repeatedly over time (e.g. on a daily basis) in order to capture and quantify this change in cognitive characteristics. EI rating engine 205 is operable to compute a periodic (e.g. weekly) cognitive change metric and EI change metric for each individual. EI rating engine 205 is operable compute a difference metric between an individual's cognitive status and/or change and an individual's EI status and/or change which provides an indication of an individual's productivity capacity (or burnout indicating a degree by which their productivity may be hindered).

Group personality assessment engine 206 is operable to compute at least one metric associated with the cognitive and/or emotional intelligence characteristics of a group of individuals (e.g. a team, workgroup, etc.). The EI characteristics and cognitive characteristics may be computed for each individual in the same fashion as described above with respect to EI rating engine 205. Group EI rating engine 206 is operable to combine the individual metrics to compute a group metric. In one aspect, group EI rating engine 206 is operable to compute a group metric for each cognitive category (as described above) and/or each emotional intelligence category (as described above). Group EI rating engine 206 is operable to factor each individual's position or team role into the group metric computation(s). In one aspect, group EI rating engine 206 applies a different weighting factor to each individual's cognitive and/or EI characteristics or metrics based on their position or team role in computing group metrics.

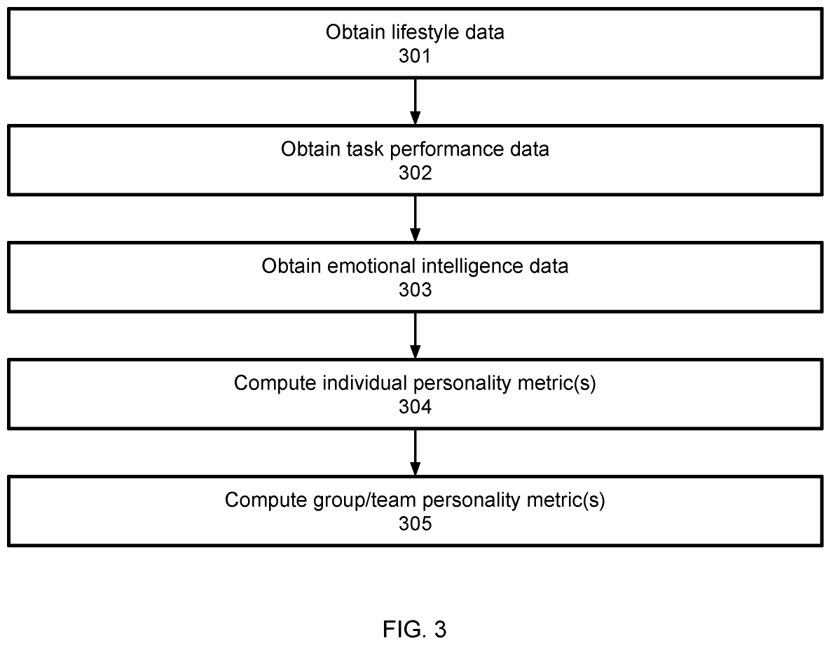

FIG. 3 illustrates an exemplary process for rating performance according to one embodiment of the invention. The process comprises obtaining lifestyle data 301, obtaining cognitive task performance data 302, obtaining emotional intelligence data 303, computing individual personality metric(s) 304, and computing group/team personality metric(s) 305. The process may comprise additional steps, fewer steps, and/or a different order of steps without departing from the scope of the invention as would be apparent to one of ordinary skill in the art.

At step 301, the process may comprise obtaining lifestyle data. Lifestyle data may be obtained at a plurality of time points throughout a day and repeated over the course of weeks, months, or years. For example, data may be obtained at a plurality of occasions comprised of a plurality of user engagements, including, but not limited to, upon an individual (or user) waking up, at breakfast, at lunch, at dinner, a cardiovascular record, a digestive system record, and a daily summary record, each of which may have a designated time or time range at which they are to occur. Lifestyle data may comprise at least one of sleep duration, bedtime, wake up time, diet information (e.g. food ingredients and portion size at each meal throughout the day, e.g. obtained at breakfast, lunch and dinner times), cardiovascular information (e.g. heart rate measurements throughout the day, calories burned, etc.), and digestive system information (e.g. bowel movement information, urine output information). Lifestyle data may be comprised of physiology data obtained from a wearable sensor. The physiology data may be associated with lifestyle activities of the user obtained over a plurality of days. The physiology data may comprise at least one of sleep data, heart rate data, and caloric expenditure data. Lifestyle data may be comprised of user reported lifestyle data. User reported lifestyle data may comprise at least one of diet data and waste excretion data. The lifestyle data may be used to compute at least one lifestyle metric. The lifestyle metric may comprise at least one of a passive lifestyle metric, an active lifestyle metric, and a biofeedback lifestyle metric.

At step 302, the process may comprise obtaining cognitive task performance data. cognitive task performance data may be obtained at a plurality of time points over the course of days, weeks, months, etc. via cognitive tasks designed to evaluate an individual's executive function under different conditions. The different conditions may comprise at least one of a baseline condition (e.g. a normal condition with no external stimuli), a mental stress condition (e.g. audio and/or visual stimuli), and a physical stress condition (e.g. during and/or after exercise or other physical exertion). The cognitive task performance data may comprise physiology data obtained from a wearable sensor (e.g. the same used to obtain the lifestyle based physiology data) in association with specific task performance activities performed by a user. The task performance activities may comprise activities which are different than the lifestyle activities. The task performance activities, and thus the corresponding physiology data, may be associated with different conditions. For example, the physiology data may be obtained during task performance activities performed under at least one of a baseline condition and a plurality of stimulus conditions. The task performance activities may be used to compute at least one cognitive performance metric. The cognitive performance metrics may comprise at least one of planning, anticipation, risk management, focus, memory, and connectivity.

At step 303, the process may comprise obtaining emotional intelligence data. Emotional intelligence data may be obtained at a plurality of time points throughout a day and repeated over the course of weeks, months, or years. Emotional intelligence data may be obtained via a plurality of engagements with a user throughout a day wherein a user is prompted to answer at least one question designed to assess mental status. For example, a user may be prompted to answer at least one question at at least one time point, the time points comprising at least one of when a user wakes up, at breakfast, at lunch, at dinner, a recording of cardiovascular information, a recording of digestive system information, and a daily summary engagement. The emotional intelligence data may be used to compute at least one emotional intelligence metric.

At step 304, the process may comprise computing individual personality metric(s). The data obtained in steps 301-303 may be used to compute at least one of active lifestyle data, passive lifestyle data, and biofeedback data. The following metrics are merely exemplary and additional metrics or variations of the metrics disclosed below using different variables may be used as would be apparent to one of ordinary skill in the art.

Active lifestyle data may comprise at least one metric for each of plurality of categories, the categories comprising at least one of bedtime, diet quality, cardiovascular, and emotional intelligence.

The bedtime metric(s) may comprise comparison of obtained bedtime information with a benchmark time in order to compute whether bedtime is maintaining consistency, trending towards the benchmark, or trending away from the benchmark. For example, a user specific benchmark may comprise a 30 day average bedtime against which the most recent bedtime data is compared.

Diet quality metric(s) may comprise comparison of obtained diet information with a benchmark diet metric (e.g. caloric intake, macronutrient breakdown, food ingredient/type ratios). For example, a moving average calculation of the percentage of food intake that is protein, fruits and vegetables over the last 30 days may be used as a benchmark against which the most recent diet data is compared.

Cardiovascular metric(s) may comprise comparison of obtained cardiovascular information with a cardiovascular benchmark. For example, the cardiovascular benchmark may comprise an expected number of calories burned such as a fixed value (e.g. 2000) or a user based metric such as the average number of daily calories consumed over a given time period (e.g. the last week, month). The calories consumed on a given day may be compared against the benchmark to determine directional trends in cardiovascular information. A similar approach may be applied to calories burned, or net caloric balance (e.g. caloric intake minus calories burned).

Emotional intelligence metric(s) may comprise comparison of emotional intelligence data against a user specific benchmark such as a moving average calculation. For example, a user specific benchmark may comprise a 30 day average derived from emotional intelligence data against which the most recent EI data is compared.

Passive lifestyle data may comprise at least one metric for each of plurality of categories, the categories comprising at least one of sleep duration, wake up time, punctuality, diet and bowel movement relationship, dinner time relative to sunset, wake up time relative to sunrise, and bedtime relative to sunset

Sleep duration metric(s) may comprise an amount of sleep recorded for each individual. In one aspect, sleep duration may comprise an average amount of sleep over a given time period. In one aspect, a sleep duration metric may comprise a comparison of the most recent sleep duration information against a benchmark, such as a fixed value (e.g. 8 hours) or a user specific sleep duration value (e.g. 30 day moving average of user specific sleep duration).

Wake up time metric(s) may comprise recorded wake up times for each individual. In one aspect, wake up metric(s) may comprise an average wake up time over a given time period. In one aspect, a wake up metric may comprise a comparison of the most recent wake up time information against a benchmark, such as a fixed value (e.g. a specified time such as 7:00 am) or a user specific wake up time (e.g. 30 day moving average of user specific wake up times) or sunrise time

Punctuality metric(s) may comprise the difference between the time when an engagement occurred and the designated time or time range when the engagement was intended to occur. Punctuality metric(s) may be computed for at least one of the daily engagements and may be computed for each of the daily engagements. In one aspect, punctuality metric(s) may comprise a comparison of the most recent punctuality information against a benchmark, such as a user specific punctuality metric (e.g. 30 day moving average of punctuality metrics).

Diet and bowel movement relationship metric(s) may comprise a measure of expected bowel movement characteristics with respect to user reported bowel movement characteristics. Expected bowel movement characteristics may be determined from user reported diet information and compared with user reported bowel movement characteristics. The comparison may reveal aspects of a user's self-awareness or identify patterns of lying or falsifying information. For example, a mismatch between the expected bowel movement characteristics derived from diet and bowel movement characteristics reported by the user indicates a lack of self-awareness and/or an indication that inaccurate information was provided by the user (e.g. lying). Alternatively, when diet based bowel movement characteristics and user reported bowel movement characteristics match or have a closer relationship, this indicates higher user self-awareness and/or accuracy in user provided information (e.g. truthfulness).

Dinner time relative to sunset metric(s) may comprise computing the duration between obtained dinner time information for a given day with sunset time for that day. These metrics may comprise obtaining weather related data including daily sunset times against which user dinner time information is compared. In one aspect, dinner time relative to sunset metric(s) may comprise a comparison of the most recent dinner to sunset time gap information against a benchmark, such as a user specific time gap metric (e.g. 30 day moving average of dinner to sunset time gap metrics).

Wake up time relative to sunrise metric(s) may comprise computing the duration between obtained wake up time information for a given day with sunrise time for that day. These metrics may comprise obtaining weather related data including daily sunrise times against which user wake up time information is compared. In one aspect, wake up time relative to sunrise metric(s) may comprise a comparison of the most recent wake up to sunrise time gap information against a benchmark, such as a user specific time gap metric (e.g. 30 day moving average of wake up to sunrise time gap metrics).

Bedtime relative to sunset metric(s) may comprise computing the duration between obtained bedtime information for a given day with sunset time for that day. These metrics may comprise obtaining weather related data including daily sunset times against which user bedtime information is compared. In one aspect, bedtime relative to sunset metric(s) may comprise a comparison of the most recent bedtime to sunset time gap information against a benchmark, such as a user specific time gap metric (e.g. 30 day moving average of bedtime to sunset time gap metrics).

Biofeedback data may comprise at least one metric for each of plurality of categories, the categories comprising at least one of heart rate measures, sleep metrics, urination characteristics, and bowel movement characteristics.

Heart rate measures may comprise heart rate characteristics for a day or a plurality of time points throughout a day. Exemplary heart rate measures comprise resting heart rate, heart rate variability, etc. Heart rate measures may comprise comparison of the most recent heart rate measures against a benchmark, such as user specific heart rate characteristics (e.g. 30 day moving average of resting heart rate, heart rate variability, etc.)

Sleep metrics may comprise sleep characteristics such as REM or deep sleep duration, percentage of total sleep which is REM or deep sleep, etc. Sleep metrics may comprise comparison of the most recent sleep information against a benchmark (e.g. 30 day moving average of sleep characteristics).

Urination characteristics may comprise characteristics indicative of user hydration status, such as urine color. Urine characteristics may comprise comparison of the most recent urine information against a benchmark (e.g. 30 day moving average of urine characteristics).

Bowel movement characteristics may comprise information associated with bowel movement type. Bowel movement characteristics may comprise comparison of the most recent bowel movement information against a benchmark (e.g. 30 day moving average of bowel movement characteristics).

The above metrics may be used in combination to determine at least one of cognitive and emotional intelligence metrics for each individual. For example, at least one cognitive function metric may be computed based on the combination of the lifestyle data, cognitive task performance data, and emotional intelligence data. The cognitive function metric may reflect a projected productivity capacity for the user. For example, at least one cognitive function metric may be computed from the combination of two or more of the lifestyle metric(s), cognitive task performance metric(s), and emotional intelligence metric(s). In one aspect, the cognitive function metric(s) may be computed using a weighted combination of the lifestyle metric(s), cognitive task performance metric(s), and emotional intelligence metric(s). In one aspect, computing at least one cognitive function metric comprises adjusting at least one emotional intelligence metric computed from user reported emotional intelligence data based on the lifestyle metric to account for user bias in self-reported data. Additional examples and the inter-relationship between emotional intelligence (true and perceived) and cognitive performance or capabilities are described below.

Certain active lifestyle choices (and thus the corresponding metrics above) and biofeedback data would impact an individual's cognitive level or capacity. Each of the above mentioned, exemplary cognitive characteristics (e.g. planning, anticipation, risk management, focus, memory, connectivity) may be affected differently by different lifestyle and biofeedback metrics. Step 304 may comprise computing a cognitive level or capacity for each of the cognitive characteristics using a combination of at least one active lifestyle metric and at least one biofeedback metric. In one aspect, computing the cognitive level or capacity may comprise a weighting algorithm (unique for each cognitive characteristic) in order to compute each individual's cognitive level or capacity for a given day and over a given time frame (e.g. weekly, monthly, etc.) for each cognitive characteristic.

Certain passive lifestyle characteristics (and thus the corresponding metrics above) would reveal an individual's true emotional intelligence which may differ from starting emotional intelligence metrics derived from questionnaire data as described above. Each of the above mentioned, exemplary emotional intelligence characteristics (e.g. motivation, self awareness, interpersonal skills, self regulation, adaptability, facial recognition) may be determined from observation of passive lifestyle characteristics over time. Step 304 may comprise computing an emotional intelligence metric for each of the emotional intelligence characteristics using a combination of the above passive lifestyle characteristics. In one aspect, computing the emotional intelligence metrics may comprise a weighting algorithm (unique for each emotional intelligence characteristic) in order to compute each individual's EI characteristics for a given day and over a given time frame (e.g. weekly, monthly, etc.). For example, two individuals may consider themselves highly motivated and thus have the same initial emotional intelligence rating derived from an initial questionnaire as described above. However, the first individual may go to bed later, wake up later, skip breakfast, and be less punctual (e.g. late or arriving just on time to the workplace or other meetings/appointments) while the second individual may go to sleep carlier, wake up earlier, exercise before eating breakfast and be more punctual (e.g. arriving 15 minutes early to the workplace or other meetings/appointments. These passive lifestyle characteristics would reveal the individuals' true motivation, such as the first individual having a true emotional intelligence rating lower than the initial questionnaire indicated while the second individual having a true emotional intelligence rating more in line with what the initial questionnaire indicated. In one aspect.

In addition to being able to compute true emotional intelligence metrics, step 304 may also comprise determining individual burnout metrics by computing the difference between an individual's cognitive capacity and emotional intelligence.

At step 305, the process may comprise computing group or team personality metrics. In one aspect, the metrics computed above for each individual on a team or part of a work group may be combined in order to determine a group or team metric. In one aspect, the individual metrics are combined using a weighting algorithm which factors each individual's position/role into each calculation. For example, a director or CEO may have a higher weight (e.g. 10/10) applied to the cognitive and EI metrics when computing combined team metrics, while an administrative assistant has a lower weight (e.g. 1/10) applied to the metrics. In one aspect, different productivity categories may be associated with different weighting factors based on the individual's role/position. For example, for strategic decision making based productivity measures, a general manager may have higher weight (e.g. 10/10) applied and an assistant may have a lower weight (e.g. 1/10) applied, while for sales based productivity measures, the general manager may have a lower weight (e.g. 2/10) applied and the assistant may have a higher weight (e.g. 10/10) applied. In addition to computing group/team cognitive and emotional intelligence metrics, step 305 may comprise computing team burnout by determining the difference between the team or groups combined cognitive metrics/capacity and emotional intelligence metrics (which again may apply the weighting factors described above).

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

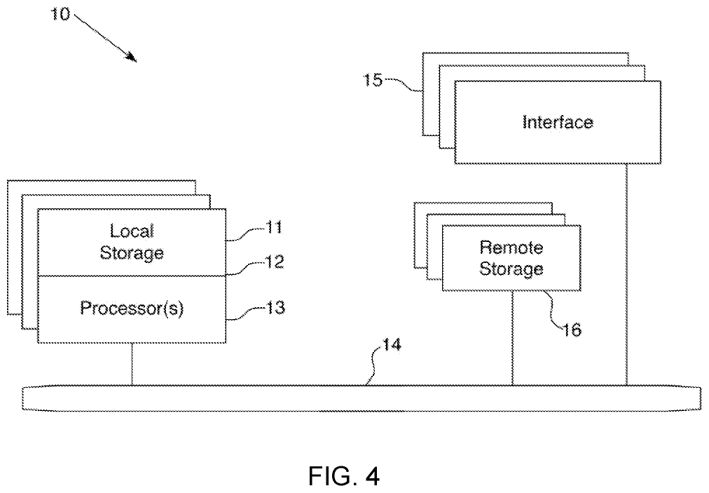

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components or the like may be and/or comprise hardware and/or software as described herein. For example, the performance rating system 103 and subcomponents thereof may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

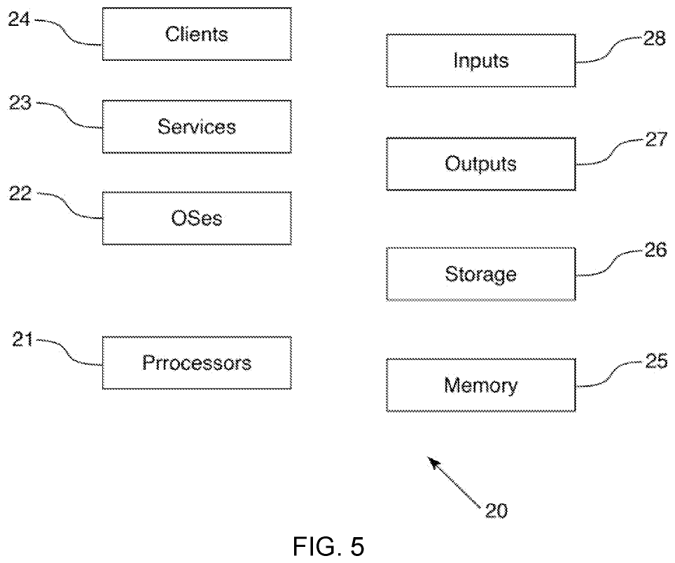

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

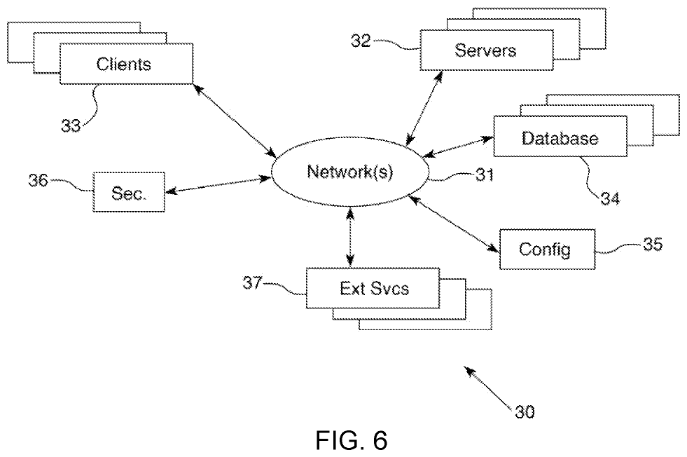

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

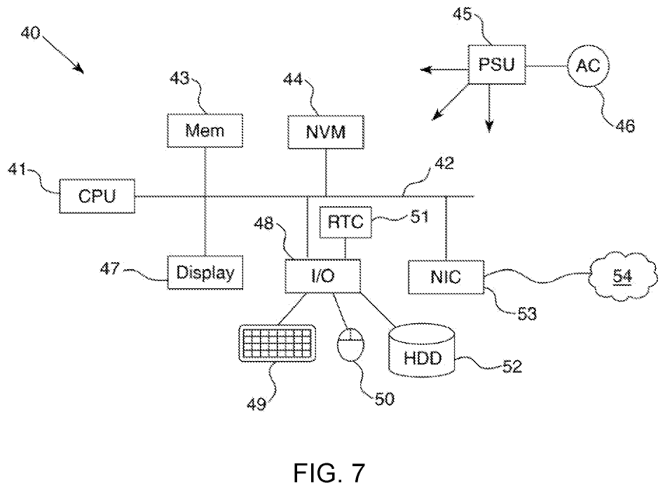

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising.” “includes,” “including.” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement. operation and details of the method and apparatus disclosed herein without departing from the spirit and scope defined in the appended claims.

What is claimed is:

1. A computer implemented method for evaluating and forecasting cognitive function metrics based on emotional intelligence characteristics of an individual, the computer implemented method comprising:
obtaining, from a wearable sensor, first physiology data associated with lifestyle activities of a user, the lifestyle activities comprising day to day activities, the first physiology data obtained over a plurality of days;
obtaining, via at least one user device, user reported lifestyle data, the user reported lifestyle data comprising user reported data obtained over a plurality of days;
computing, using at least one of the first physiology data and the user reported lifestyle data, at least one lifestyle metric;
obtaining, from the wearable sensor, second physiology data associated with specific task performance activities of a user, wherein the task performance activities are different than the lifestyle activities, wherein the second physiology data associated with specific task performance activities is obtained during a baseline condition and a plurality of stimulus conditions;
computing, using the second physiology data, at least one cognitive performance metric;
obtaining, via at least one user device, emotional intelligence data, the emotional intelligence data comprising user responses to a plurality of questions;
computing, using the emotional intelligence data, at least one emotional intelligence metric;
computing at least one cognitive function metric using the at least one lifestyle metric, the at least one cognitive function metric and the at least one emotional intelligence metric; and
providing an indication of the at least one cognitive function metric to at least one of the user or at least one member of a team of individuals associated with the user.

2. The computer implemented method according to claim 1, wherein the first physiology data comprises at least one of sleep data, heart rate data, and caloric expenditure data.

3. The computer implemented method according to claim 1, wherein the user reported lifestyle data comprises at least one of diet data and waste excretion data.

4. The computer implemented method according to claim 1, wherein the at least one lifestyle metric comprises at least one of a passive lifestyle metric, an active lifestyle metric, and a biofeedback lifestyle metric.

5. The computer implemented method according to claim 1, wherein the at least one cognitive performance metric comprises at least one of planning, anticipation, risk management, focus, memory, and connectivity.

6. The computer implemented method according to claim 1, wherein the at least one emotional intelligence metric comprises at least one of motivation, self awareness, interpersonal skill, self regulation, adaptability, and facial recognition.

7. The computer implemented method according to claim 1, wherein the at least one cognitive function metric reflects a projected productivity capacity for the user.

8. The computer implemented method according to claim 1, wherein providing an indication of cognitive function comprises providing an indication of reduced cognitive indicative of an expected reduced productivity capacity.

9. The computer implemented method according to claim 1, further comprising adjusting the at least one emotional intelligence metric computed from user reported emotional intelligence data based on the lifestyle metric to account for user bias in self-reported data.

10. The computer implemented method according to claim 1, further comprising computing a projected team performance metric by combining the computed metrics for a plurality of users.

11. The computer implemented method according to claim 10, wherein the projected team performance metric is computed by applying different weighting factors to the computed metrics for the plurality of users based on the role or position of the user within the team.

12. A computing system for evaluating and forecasting cognitive function metrics based on emotional intelligence characteristics of an individual, the computing system comprising:
at least one computing processor; and
memory comprising instructions that, when executed by the at least one computing processor, enable the computing system to:
obtain, from a wearable sensor, first physiology data associated with lifestyle activities of a user, the lifestyle activities comprising day to day activities, the first physiology data obtained over a plurality of days;
obtain, via at least one user device, user reported lifestyle data, the user reported lifestyle data comprising user reported data obtained over a plurality of days;
compute, using at least one of the first physiology data and the user reported lifestyle data, at least one lifestyle metric;
obtain, from the wearable sensor, second physiology data associated with specific task performance activities of a user, wherein the task performance activities are different than the lifestyle activities, wherein the second physiology data associated with specific task performance activities is obtained during a baseline condition and a plurality of stimulus conditions;
compute, using the second physiology data, at least one cognitive performance metric;
obtain, via at least one user device, emotional intelligence data, the emotional intelligence data comprising user responses to a plurality of questions;
compute, using the emotional intelligence data, at least one emotional intelligence metric;
compute at least one cognitive function metric using the at least one lifestyle metric, the at least one cognitive function metric and the at least one emotional intelligence metric; and
provide an indication of the at least one cognitive function metric to at least one of the user or at least one member of a team of individuals associated with the user.

13. A computer readable medium comprising instructions that when executed by a processor enable the processor to execute a method for evaluating and forecasting cognitive function metrics based on emotional intelligence characteristics of an individual, the method comprising:
obtaining, from a wearable sensor, first physiology data associated with lifestyle activities of a user, the lifestyle activities comprising day to day activities, the first physiology data obtained over a plurality of days;
obtaining, via at least one user device, user reported lifestyle data, the user reported lifestyle data comprising user reported data obtained over a plurality of days;
computing, using at least one of the first physiology data and the user reported lifestyle data, at least one lifestyle metric;
obtaining, from the wearable sensor, second physiology data associated with specific task performance activities of a user, wherein the task performance activities are different than the lifestyle activities, wherein the second physiology data associated with specific task performance activities is obtained during a baseline condition and a plurality of stimulus conditions;
computing, using the second physiology data, at least one cognitive performance metric;
obtaining, via at least one user device, emotional intelligence data, the emotional intelligence data comprising user responses to a plurality of questions;
computing, using the emotional intelligence data, at least one emotional intelligence metric;
computing at least one cognitive function metric using the at least one lifestyle metric, the at least one cognitive function metric and the at least one emotional intelligence metric; and
providing an indication of the at least one cognitive function metric to at least one of the user or at least one member of a team of individuals associated with the user.
